(19) United States
(12) Patent Application Publication
(10) Pub. No.: US 2011/0294192 A1
(43) Pub. Date: Dec. 1, 2011

(54) FUSION COLLAGENASE IN WHICH AFFINITY TAG IS LINKED AND METHOD FOR PRODUCING THE SAME

(75) Inventors: Takayoshi Fukushima, Kanagawa (JP); Kengo Yokoyama, Kanagawa (JP); Koichiro Murashima, Kanagawa (JP); Masafumi Goto, Kanagawa (JP); Youhei Yamagata, Miyagi (JP)

(73) Assignees: TOHOKU UNIVERSITY, Sendai-shi, Miyagi (JP); MEIJI SEIKA PHARMA CO., LTD., Chuo-ku, Tokyo (JP)

(21) Appl. No.: 13/129,969

(22) PCT Filed: Nov. 6, 2009
(86) PCT No.: PCT/JP2009/068968

§ 371 Date: Aug. 12, 2011

(30) Foreign Application Priority Data
Nov. 19, 2008 (JP) 2008-295922

(51) Int. Cl. C12N 9/52

(52) U.S. Cl. 435/220

(57) ABSTRACT

A fusion collagenase in which an affinity tag is added to the carboxyl terminal of a collagenase was expressed as a recombinant protein. It was found that a collagenase having a collagen-binding domain can be selectively collected by purifying the obtained fusion collagenase by affinity chromatography.

### TECHNICAL FIELD

The present invention relates to a collagenase used for isolating cells (cell mass) from an organ, such as cells of pancreatic islets from a pancreas.

### BACKGROUND ART

As a curative therapy for diabetes, pancreatic islet transplantation has been known, in which pancreatic islets (insulin-producing cells) isolated from a pancreas treated with a protease are transplanted into the portal vein of a diabetic patient via intraverous drip. This transplantation method does not require laparotomy for the patient during the transplantation, and therefore recently has attracted attention as a curative therapy, which is safe and simple, for diabetes.

Pancreatic islets are isolated from a pancreas by treating the pancreas with a collagenase and a neutral metalloprotease. As the collagenase used for this purpose, a collagenase derived from Clostridium histolyticum is particularly effective (Non-Patent Literatures 1, 2).

The collagenase derived from Clostridium histolyticum has been known to include two types of enzyme having different substrate specificity; collagenase G and collagenase H (Non-Patent Literatures 1, 2). Genes encoding collagenase G and collagenase H have been already isolated, and both collagenases have been found to be multi-domain enzymes including a catalytic domain on the amino terminal side and a collagen-binding domain (hereinafter, referred to as “CBD”) on the carboxyl terminal side (Non-Patent Literatures 1, 2).

However, there has been a problem that, when a collagenase is produced by Clostridium histolyticum, part of the expressed collagenase is degraded (Non-Patent Literature 2), and treating of a pancreas with a collagenase mixed with the degraded collagenase decreases the quality of isolated pancreatic islets. Thus, for isolation of pancreatic islets, it is desirable to treat a pancreas with a collagenase obtained by removing such a degraded collagenase as much as possible therefrom.

However, a non-degraded collagenase and a degraded collagenase are similar in physicochemical properties, and therefore it has been difficult to separate these collagenases by methods such as ion-exchange chromatography or hydrophobic chromatography.

In such backgrounds, a method has been desired for preparing a collagenase by selectively collecting a non-degraded collagenase from collagenases derived from Clostridium histolyticum.

It should be noted that degradation of a collagenase derived from Clostridium histolyticum has not been reported yet with the collagenase being expressed as a recombinant protein in a host such as E. coli.

### CITATION LIST

### Non Patent Literatures

[NPL 1] Yoshihara, K. et. al. Journal of Bacteriology. (1994), 176, 6489-6496
[NPL 2] Matsushita, O. et. al. Journal of Bacteriology. (1999), 181, 923-933

### SUMMARY OF INVENTION

### Technical Problem

The present invention aims at selectively collecting a non-degraded collagenase by removing a degraded collagenase from collagenases derived from Clostridium histolyticum.

### Solution to Problem

With respect to collagenases derived from Clostridium histolyticum, fusion collagenases in which affinity tags linked to the carboxyl terminals of the collagenases were expressed as recombinant proteins by the present inventors. When a collagenase derived from Clostridium histolyticum is expressed as a recombinant protein in a host such as E. coli, it has not been well-predicted whether the recombinant protein is degraded by the action of a protease in a host or not, and how the recombinant protein is degraded supposing that it is degraded. The present inventors, however, purified the fusion protein expressed in a host with an affinity column, and accidentally found that a degraded collagenase was removed and that a single collagenase having a collagenase activity was selectively collected. This is based on the following assumptions that: a collagenase derived from Clostridium histolyticum expressed in a host was degraded at the CBD in the neighborhood of the fused affinity tag, and therefore degradation of the CBD allowed the affinity tag to be separated from the collagenase; and in the purification stage by affinity chromatography, the degraded collagenase was not adsorbed to the affinity column but only a non-degraded collagenase was adsorbed to the affinity column.

In other words, the present inventors found out that if an affinity tag is linked to a collagenase in a specific configuration, a single collagenase having a collagenase activity can be selectively collected in the affinity purification process without the collagenase degraded in a host being adsorbed. This discovery has led the inventors to the completion of the present invention.

The present invention relates to inventions as follows.

<1> A fusion collagenase in which an affinity tag is directly or indirectly linked to a collagenase, wherein the collagenase and the affinity tag are linked to each other in such a manner that a fragment having a collagenase activity and the affinity tag are separated from each other when the fusion collagenase expressed in a host is degraded by an action of the host.

<2> The fusion collagenase according to <1>, wherein the affinity tag is directly or indirectly linked to a carboxyl terminal of the collagenase.

<3> The fusion collagenase according to <2>, wherein the affinity tag is two or more consecutive histidine residues.

<4> The fusion collagenase according to <1>, wherein the collagenase is derived from Clostridium histolyticum.

<5> The fusion collagenase according to <1>, wherein an amino acid sequence of the collagenase is selected from the following (i), (ii), (iii) and (iv):

(i) a collagenase comprising all or part of an amino acid sequence from positions −110 to 1008 of any one of SEQ ID NOS: 1 and 2;

(ii) a collagenase comprising an amino acid sequence from positions −110 to 1008 of any one of SEQ ID NOS: 1 and 2 in which one or more amino acids are deleted, substituted, inserted, or added;

(iii) a collagenase comprising an amino acid sequence having a homology of 70% or more with the amino acid sequence from positions −110 to 1008 of any one of SEQ ID NOS: 1 and 2; and

(iv) a collagenase in which all or part of an amino acid sequence from positions −110 to −1 of any one of SEQ ID NOS: 1 and 2 is removed from any one of the collagenases described in (i) to (iii).

<6> The fusion collagenase according to <5>, comprising all or part of an amino acid sequence from positions −110 to 1021 of SEQ ID NO: 3.

<7> The fusion collagenase according to <6>, from which all or part of an amino acid sequence from positions −110 to −1 of SEQ ID NO: 3 is removed.

<8> The fusion collagenase according to >1≦, wherein an amino acid sequence of the collagenase is selected from the following (i), (ii), (iii) and (iv):

(i) a collagenase comprising all or part of an amino acid sequence from positions −40 to 981 of any one of SEQ ID NOS: 5 and 6;

(ii) a collagenase comprising an amino acid sequence from positions −40 to 981 of any one of SEQ ID NOS: 5 and 6 in which one or more amino acids are deleted, substituted, inserted, or added;

(iii) a collagenase comprising an amino acid sequence having a homology of 70% or more with the amino acid sequence from positions −40 to 981 of any one of SEQ ID NOS: 5 and 6; and

(iv) a collagenase in which all or part of an amino acid sequence from positions −40 to −1 of any one of SEQ ID NOS: 5 and 6 is removed from any one of the collagenases described in (i) to (iii).

<9> The fusion collagenase according to <8>, comprising all or part of an amino acid sequence from positions −40 to 994 of SEQ ID NO: 7.

<10> The fusion collagenase according to >9≦, from which all or part of an amino acid sequence from positions −40 to −1 of SEQ ID NO: 7 is removed.

<11> A DNA encoding the fusion collagenase according to any one of <1> to <10>.

<12> A DNA comprising a base sequence from positions 1 to 3396 of SEQ ID NO: 4.

<13> A DNA comprising a base sequence from positions 1 to 3105 of SEQ ID NO: 8.

<14> An expression vector comprising the DNA according to any one of <11> to <13>.

<15> A host cell transformed by any one of the DNA according to any one of <11> to <13> and the expression vector according to <14>.

<16> The host cell according to <15>, wherein the host cell is E. coli.

<17> A method for producing a fusion collagenase, wherein a culture obtained by culturing the host cell according to any one of <15> and <16> is purified by affinity chromatography corresponding to an affinity tag to thereby selectively collect a fusion collagenase having a collagen-binding domain.

<18> A fusion collagenase produced by the method according to <17>.

### Advantageous Effect of Invention

The present invention provides a fusion collagenase in which an affinity tag is linked to a collagenase derived from Clostridium histolyticum, wherein the collagenase and the affinity tag are linked to each other in such a manner that a fragment having a collagenase activity and the affinity tag are separated from each other when the fusion collagenase expressed in a host is degraded by an action of the host. The present invention also provides a DNA required to produce the fusion collagenase as a recombinant protein efficiently, and a host cell expressing the fusion collagenase as a recombinant protein. In addition, the present invention provides a method capable of selectively collecting a single fusion collagenase having a CBD by removing a collagenase in which part or all of a CBD is degraded from a culture solution obtained by culturing a host cell expressing the fusion collagenase. The present invention leads to efficient production of a fusion collagenase having a CBD.

### BRIEF DESCRIPTION OF DRAWINGS

FIG. 1 is a drawing showing a structure of a plasmid pColG. In the drawing, the meanings of the symbols are as follows (the oblique types in the drawing are underlined). colG: a collagenase G gene, PlacZ: a lacZ promoter, and Amp^{r}: an ampicillin-resistance gene.

FIG. 2 is a drawing showing a structure of a plasmid pColG-His. In the drawing, the meanings of the symbols are as follows (the oblique types in the drawing are underlined). colG: a collagenase G gene, PlacZ: a lacZ promoter, Amp^{r}: an ampicillin-resistance gene, MCS: a multi cloning site, and 6XHis: a histidine tag.

FIG. 3 is a picture of electrophoresis showing the result of activity staining performed on an extract of E. coli having fusion collagenase G expressed.

FIG. 4 is a picture of electrophoresis showing the result of activity staining performed on a solution of fusion collagenase G subjected to affinity chromatography.

FIG. 5 is a drawing showing a structure of a plasmid pColH. In the drawing, the meanings of the symbols are as follows (the oblique types in the drawing are underlined). colH: a collagenase H gene, PlacZ: a lacZ promoter, and Amp^{r}: an ampicillin-resistance gene.

FIG. 6 is a drawing showing a structure of a plasmid pColH-His. In the drawing, the meanings of the symbols are as follows (the oblique types in the drawing are underlined). colH: a collagenase H gene, PlacZ: a lacZ promoter, Amp^{r}: an ampicillin-resistance gene, MCS: a multi cloning site, and 6XHis: a histidine tag.

FIG. 7 is a picture of electrophoresis showing the result of activity staining performed on an extract of E. coli having fusion collagenase H expressed.

FIG. 8 is a picture of electrophoresis showing the result of activity staining performed on a solution of fusion collagenase H subjected to affinity chromatography.

### DESCRIPTION OF EMBODIMENTS

(Fusion Collagenase)

In the present invention, a fusion collagenase refers to a protein in which an affinity tag is directly or indirectly linked to a collagenase, i.e. a fusion collagenase in which the collagenase and the affinity tag are linked to each other in such a manner that a fragment having a collagenase activity and the affinity tag in the fusion collagenase are separated from each other when the fusion collagenase expressed in a host is degraded by an action of the host. The linking manner of such a collagenase to the affinity tag is preferably linking to a CBD of the collagenase, and most preferably linking to the carboxyl terminal of the CBD (i.e. the carboxyl terminal of the collagenase). In the present invention, the “CBD” means regions of segments 3a and 3b in collagenase G (position 776 to carboxyl terminal of each of SEQ ID NOS: 1 and 2), and a region of a segment 3 in collagenase H (from position 864 to carboxyl terminal of SEQ ID NO: 5) (Non-Patent Literature 2).

As a collagenase in the present invention, any collagenase can be used as long as pancreatic islets can be isolated from a pancreas by being treated in combination with a neutral metalloprotease. Particularly, the use of a collagenase derived from Clostridium histolyticum is desirable. Any collagenase derived from Clostridium histolyticum can be used, but particularly the use of the collagenase G of any one of SEQ ID NOS: 1 and 2, or the collagenase H of any one of SEQ ID NOS: 5 and 6 is desirable. In addition, such a collagenase may include an amino acid sequence having all or part of a signal sequence removed.

Here, the amino acid sequence of the collagenase G may be any of the followings as long as the collagenase activity and the binding to collagen are retained: (i) a collagenase comprising all or part of an amino acid sequence from positions −110 to 1008 of any one of SEQ ID NOS: 1 and 2; (ii) a collagenase comprising an amino acid sequence from positions −110 to 1008 of any one of SEQ ID NOS: 1 and 2 in which one or more amino acids are deleted, substituted, inserted, or added; (iii) a collagenase comprising an amino acid sequence having a homology of 70% or more with the amino acid sequence from positions −110 to 1008 of any one of SEQ ID NOS: 1 and 2; and (iv) a collagenase in which all or part of a signal sequence including an amino acid sequence from positions −110 to −1 of any one of SEQ ID NOS: 1 and 2 is removed from any one of the collagenases described in (i) to (iii).

Similarly, the amino acid sequence of the collagenase H may be any of the followings as long as the collagenase activity and the binding to collagen are retained: (i) a collagenase comprising all or part of an amino acid sequence from positions −40 to 981 of any one of SEQ ID NOS: 5 and 6; (ii) a collagenase comprising an amino acid sequence from positions −40 to 981 of any one of SEQ ID NOS: 5 and 6 in which one or more amino acids are deleted, substituted, inserted, or added; (iii) a collagenase comprising an amino acid sequence having a homology of 70% or more with the amino acid sequence from positions −40 to 981 of any one of SEQ TD NOS: 5 and 6; and (iv) a collagenase in which all or part of a signal sequence including an amino acid sequence from positions −40 to −1 of any one of SEQ ID NOS: 5 and 6 is removed from any one of the collagenases described in (i) to (iii).

Here, the “amino acid sequence in which one or more amino acids are deleted, substituted, inserted, or added” means that the amino acid sequence is modified by well-known methods such as site-directed mutagenesis, or substitution or the like of amino acids as many as the number of amino acids naturally substituted. The number of modification of amino acids is preferably 1 to 50, more preferably 1 to 30, further preferably 1 to 10, still further preferably 1 to 5, and most preferably 1 to 2. An example of the modified amino acid sequence preferably can be an amino acid sequence having one or more (preferably, 1 to several, or 1, 2, 3, or 4) conservative substitutions of amino acids thereof. Here, the “conservative substitution” means that at least one amino acid residue is substituted with another chemically similar amino acid residue. Examples include a case where a certain hydrophobic residue is substituted with another hydrophobic residue, a case where a certain polar residue is substituted with another polar residue having the same charge, or other cases. Functionally similar amino acids which can be subjected to such substitution are known in the art for each amino acid. Specific examples of non-polar (hydrophobic) amino acids include alanine, valine, isoleucine, leucine, proline, tryptophan, phenylalanine, methionine, etc. Specific examples of polar (neutral) amino acids include glycine, serine, threonine, tyrosine, glutamine, asparagine, cysteine, etc. Specific examples of positively-charged (basic) amino acids include arginine, histidine, lysine, etc. Furthermore, specific examples of negatively-charged (acidic) amino acids include aspartic acid, glutamic acid, etc.

Further, the “amino acid sequence having a homology of 70% or more” can be an amino acid sequence having a homology of, preferably 80% or more, more preferably 85% or more, further preferably 90% or more, still further preferably 95% or more, particularly preferably 98% or more, and most preferably 99% or more. The term “homology” with regard to base sequence or amino acid sequence is used as a meaning of the degree of correspondence, between sequences to be compared, in bases or amino acid residues constituting each sequence. Each of the numerical values of “homology” described in the present description may be any numerical value calculated using a homology search program known to those skilled in the art, and can be easily calculated by using the default (initial setting) parameters in FASTA, BLAST, etc., for example.

In the present invention, as the affinity tag to be directly or indirectly fused to the collagenase, any affinity tag can be used as long as it can be selectively bound to a certain type of carrier. For example, a histidine tag including two or more consecutive histidine residues that are selectively bound to a nickel chelate column described in Japanese Patent No. 2686090, a cellulose-binding domain that is selectively bound to an insoluble cellulose, a maltose-binding domain that is selectively bound to a maltose-binding resin, and the like can be used. Particularly, the use of the histidine tag having a smaller molecular weight is desirable.

In the present invention, the affinity tag is directly or indirectly linked to the collagenase. Here, when the collagenase and the affinity tag are indirectly linked to each other, the amino acid sequence intervening therebetween may be any sequence as long as it does not materially inhibit the activity of the fusion collagenase and the binding to collagen.

Examples of the fusion collagenase that meets the above requirements include a fusion collagenase comprising all of an amino acid sequence of SEQ ID NO: 3 in which the histidine tag is linked to the carboxyl terminal of the collagenase G, and a fusion collagenase comprising all of an amino acid sequence of SEQ ID NO: 7 in which the histidine tag is linked to the carboxyl terminal of the collagenase H. Also, the examples include a fusion collagenase comprising part of such amino acid sequence as long as the collagenase activity, the binding to collagen, and the linking to the affinity tag are retained.

(DNA Encoding Amino Acid Sequence of Fusion Collagenase)

In the present invention, a DNA encoding the fusion collagenase may be a DNA comprising any base sequences as long as the DNA encodes the amino sequence of the fusion collagenase described above.

The DNA encoding the fusion collagenase of the present invention can be obtained by artificial chemical synthesis. Also, the DNA encoding the fusion collagenase can be obtained by constructing a DNA encoding the collagenase and a DNA encoding the affinity tag separately and linking them together. In this case, the collagenase gene can be amplified by PCR using a primer synthesized based on the sequence of the gene and with a template of a DNA, such as genomic DNA, cDNA, and plasmid, including the gene. For example, the collagenase G or the collagenase H derived from Clostridium histolyticum can be amplified by PCR using a primer designed based on the sequence of 5′ end and 3′ end of the sequence of the collagenase G gene described in Non-Patent Literature 1 or the collagenase H gene described in Non-Patent Literature 2 and with a template of a genomic DNA derived from Clostridium histolyticum. Moreover, the DNA encoding the affinity tag can be amplified by PCR with a template of a genomic DNA, cDNA, plasmid, or the like including the gene encoding the affinity tag. For example, a DNA encoding the histidine tag can be amplified by PCR with a template of pET-24a(+), a commercially available vector.

Examples of the DNA that meets the above requirements include a DNA comprising all or part of the base sequence of SEQ ID NO: 4 encoding the fusion collagenase in which the histidine tag is linked to the carboxyl terminal of the collagenase G; and a DNA comprising all or part of the base sequence of SEQ ID NO: 8 for linking the histidine tag to the carboxyl terminal of the collagenase H.

(Expression Vector, and Host Cell Transformed by the Expression Vector)

The present invention provides an expression vector comprising the DNA encoding the amino acid sequence of the fusion collagenase described above, being capable of replicating the DNA in a host cell, and comprising a protein encoded by the DNA sequence in an expressible state. The present expression vector can be constructed based on a self-replicating vector, i.e., for example, a plasmid that exists as an independent extrachromosomal element and replicates independently of the replication of the chromosome. In addition, the present expression vector may be any vector that is introduced into a host cell, then incorporated into the genome of the host cell, and replicated together with the chromosome incorporated therewith. As a procedure and a method for constructing the vector according to the present invention, any procedure and any method commonly used in the field of genetic engineering can be used.

The expression vector according to the present invention, to express the fusion collagenase when the expression vector is actually introduced into the host cell, desirably comprises, in addition to the DNA encoding the amino acid sequence of the fusion collagenase described above, a DNA sequence for regulating the expression of the DNA, a genetic marker for selecting the transformed host cell, and the like. The DNA sequence for regulating the expression includes a DNA sequence encoding a promoter, terminator, and signal peptide, and the like. The promoter is not particularly limited as long as it exhibits the transcriptional activity in a host cell, and can be obtained as the DNA sequence for regulating the expression of the gene encoding either a homogeneous or heterogeneous protein with respect to the host cell. In addition, the signal peptide is not particularly limited as long as it contributes to secretion of the protein in the host cell, and can be obtained from the DNA sequence derived from the gene encoding either a homogeneous or heterogeneous protein with respect to the host cell. Moreover, the genetic marker in the present invention may be appropriately selected depending on the method for selecting the transformant, and a gene encoding for drug resistance and a gene complementing the auxotrophy, for example, can be used.

Further, according to the present invention, a host cell transformed by this expression vector is provided. This host-vector system is not particularly limited, and a system using E. coli, actinomycete, yeast, filamentous fungus, animal cell, etc., for example, or the like can be used. Particularly, E. coli is desirably used as a host.

Also, the host cell can be transformed by such expression vectors according to methods commonly used in the art.

(Culturing of Host Cell Expressing Fusion Collagenase, and Selective Collection of Fusion Collagenase Having CBD by Affinity Chromatography)

In the present invention, the fusion collagenase can be obtained from a culture which is obtained by culturing the host cell expressing the fusion collagenase can be cultured in a suitable medium. The culturing and its condition for the host cell expressing the fusion collagenase may be substantially the same as those for the host cell used.

The fusion collagenase secreted in the host cell expressing the fusion collagenase or in the culture solution can be collected by adopting methods commonly used in the art.

The fusion collagenase collected according to the above method is subjected to affinity chromatography to remove a degraded collagenase, thereby selectively collecting the fusion collagenase having the CBD. Affinity chromatography used in this case needs to be a method which corresponds to the affinity tag linked to the collagenase. For example, when the histidine tag is linked to the collagenase, a nickel chelate column or the like to which the histidine tag is selectively linked is used for purification.

The extent to which a degraded collagenase is removed by affinity chromatography can be evaluated by subjecting the fusion collagenase solution to electrophoresis using gel with gelatin added thereto and then to activity staining.

(Other Aspects)

According to the knowledge of the present inventors that the expression of a recombinant collagenase by E. coli or the like causes part or all of its CBD to be degraded, the degraded collagenase can be eliminated and a non-degraded recombinant collagenase can be specifically affinity-purified using an antibody that binds to the CBD of the recombinant collagenase. In this case, it is advantageous in that there is no need to fuse the affinity tag to the recombinant collagenase. Specifically, the present invention also provides a method for producing a collagenase, characterized in that a culture obtained by culturing a host cell having a recombinant collagenase expressed is purified with an antibody that binds to a CBD of the recombinant collagenase to thereby selectively collect a collagenase having the CBD. In addition, because the CBD and collagen have an affinity for each other, the recombinant collagenase can be specifically affinity-purified using collagen instead of the antibody described above. Specifically, the present invention also provides a method for producing a collagenase, wherein a culture obtained by culturing a host cell having a recombinant collagenase expressed is purified with collagen to thereby selectively collect a collagenase having the CBD.

### EXAMPLES

The present invention will be more specifically described by way of Examples, but the present invention is not to be limited to Examples below but is still within the gist of the present invention.

### Example 1

### Expression of Fusion Collagenase (Fusion Collagenase G) in which Histidine Tag is Linked to Carboxyl Terminal of Collagenase G Derived from Clostridium histolyticum

(1-1) Preparation of Collagenase G Gene Fragment

A collagenase G gene derived from Clostridium histolyticum was amplified from the 5′ end to the 3′ end thereof by PCR with a template of a genomic DNA derived from Clostridium histolyticum. In this case, the primers were designed so that the 3′ end of the amplified collagenase G gene served as an XbaI-recognition sequence and further that a BamHI-recognition sequence was added subsequent to the stop codon of the gene. As a result, mutations were introduced into the amino acid sequence at two positions (amino acids at positions 1007 and 1008 of SEQ ID NO: 1) on the carboxyl terminal side of natural collagenase G described in Non-Patent Literature 1. Thus, the amino acid sequence was modified to the amino acid sequence of SEQ ID NO: 2.

The primers used in this PCR were as follows.

| colG-F: | |
|---|---|
| ATGAAAAAAAATATTTTAAAGATTC | (SEQ ID NO: 9) |
| colG-R: | |
| CCGGATCCTATCTAGATACCCTTAACT | (SEQ ID NO: 10) |

The amplified collagenase G gene fragment was digested with BamHI.

(1-2) Preparation of DNA Fragment Including Region Encoding Histidine Tag

To prepare a DNA encoding the histidine tag including six consecutive histidine residues, the DNA encoding the histidine tag was amplified by PCR with a template of pET-24a(+), a commercially available vector. In the DNA fragment, in addition to the DNA encoding the histidine tag, a multi cloning site originated from the vector and a gene fragment corresponding to a T7 terminator were included. The primers were designed to include the XbaI-recognition sequence at the 5′ end of the amplified DNA fragment and the BamHI-recognition sequence at the 3′ end thereof.

The primers used in this PCR were as follows.

His-F:

| | |
|---|---|
| GCTCTAGAAAGCTTGCGGCCGCACTCGA | (SEQ ID NO: 11) |

His-R:

| | |
|---|---|
| CGGGATCCGGATATAGTTCCTCCT | (SEQ ID NO: 12) |

The amplified DNA fragment including the region encoding the histidine tag was double-digested with XbaI and BamHI.

(1-3) Preparation of lacZ Promoter Fragment

A lacZ promoter was prepared as a promoter for expressing the fusion collagenase. The DNA fragment was amplified by PCR with a template of pUC19. In this case, the primers were designed to include a HindIII-recognition sequence at the 5′ end of the amplified DNA fragment.

The primers used in this PCR were as follows.

lac-F:

| | |
|---|---|
| CCGGCAAGCTTGCCCAATACGCAAACCG | (SEQ ID NO: 13) |

lac-R:

| | |
|---|---|
| AGCTGTTTCCTGTGTGAA | (SEQ ID NO: 14) |

The amplified lacZ promoter fragment was digested with HindIII.

(1-4) Construction of Expression Vector for Fusion Collagenase G to which Histidine Tag is Linked

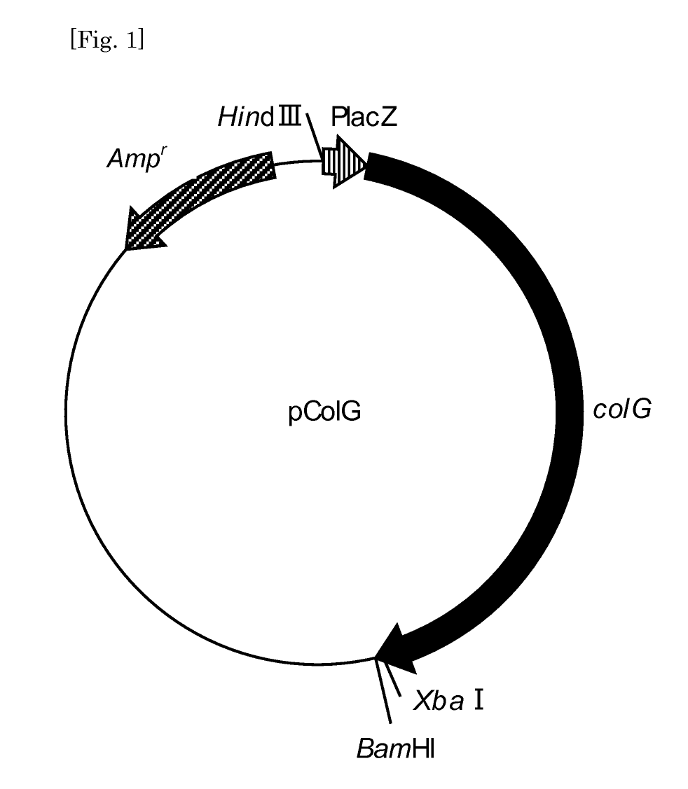

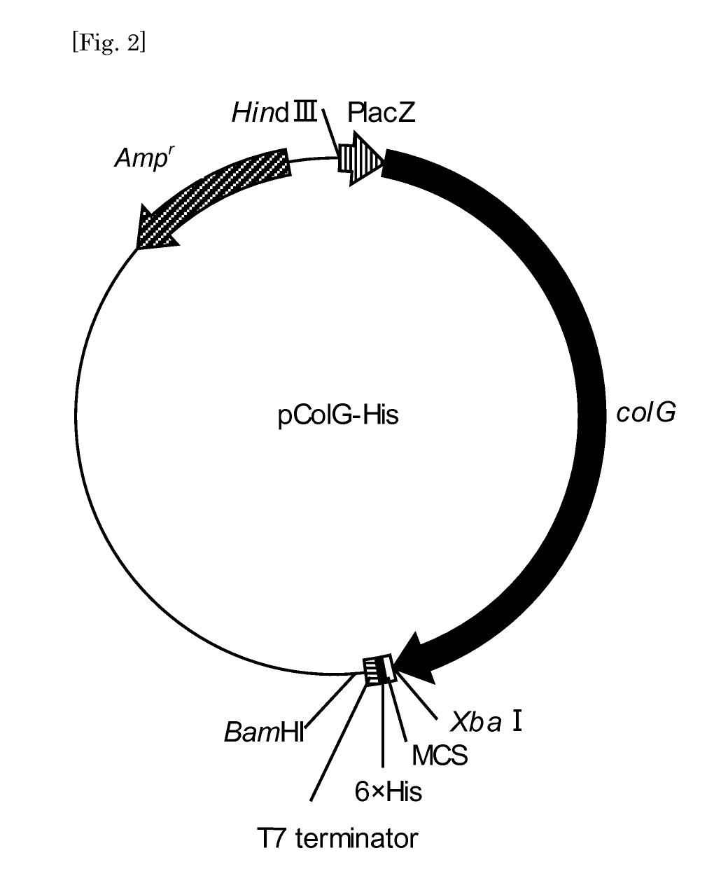

Three kinds of DNA fragments prepared by the method described above were inserted into pBR322, a commercially available vector, so as to link them in order of the lacZ promoter, the collagenase G gene, and the DNA fragment including the histidine tag from the 5′ end. First, the lacZ promoter and the collagenase G gene were inserted into pBR322. Specifically, the lacZ promoter region and the collagenase G gene fragment prepared as described above were phosphorylated, and then inserted into pBR322 that had been double-digested with HindIII and BamHI to construct pColG (FIG. 1). It is to be noted that the lacZ gene promoter region (PlacZ) and the colG gene were linked to each other at their blunt ends. Subsequently, the DNA encoding the histidine tag prepared by the method described above was inserted into pColG that had been double-digested with XbaI and BamHI to construct pColG-His (FIG. 2). The DNA that was finally inserted into pBR322 was a DNA having the base sequence from positions 1 to 3396 of Sequence Listing: 4.

(1-5) Preparation of E. coli Expressing Fusion Collagenase G

pColG-His was transformed into E. coli, Escherichia coli strain χ1776, according to the conventional method, and cultured at 37° C. for a full day on LB agar to which 20 μg/ml of diaminopimelic acid, 100 μg/ml of thymidine, and μg/ml of ampicillin had been added. Thus, E. coli expressing the fusion collagenase G was prepared.

### Example 2

### Culturing of E. coli Expressing Fusion Collagenase G, and Selective Collection of Collagenase G Having CBD

(2-1) Culturing of E. coli Expressing Fusion Collagenase G

The E. coli expressing the fusion collagenase G obtained in Example 1 was inoculated in a 250-ml Erlenmeyer flask with 100 ml of a medium being added, and cultured with stirring at 200 rpm at 28° C. for 16 hours. The medium used in this culturing was a TB medium (1.2% triptone, 2.4% yeast extract, 0.94% dipotassium hydrogenphosphate, 0.22% potassium dihydrogenphosphate, 0.8% glycerol) to which 100 μg/ml of diaminopimelic acid, 20 μg/ml of thymidine, 50 μg/ml of ampicillin, and 0.1 mM of IPTG was added.

(2-2) Preparation of Extract of E. coli Expressing Fusion Collagenase G

The resulting culture solution obtained in (2-1) was subjected to centrifugation to collect cells, followed by lysis of the collected cells in 10 ml of POP culture Regent (manufactured by Merck & Co., Inc.) to extract a protein in the cells. The supernatant obtained by centrifugation of the lysate was filtrated with a 0.2-μm membrane to remove a gene recombinant, thereby providing an extract of the E. coli expressing the fusion collagenase G.

(2-3) Selective Collection of Fusion Collagenase G Having CBD by Affinity Chromatography

To remove a degraded collagenase from the extract of the E. coli expressing the fusion collagenase G obtained in (2-2), the extract was fractionated by a nickel chelate column which was affinity chromatography for the histidine tag. To 10 ml of the extract of the E. coli expressing the fusion collagenase G prepared by the method described above, 60 ml of a buffer (20 mM sodium phosphate buffer (pH 7.5) to which 0.5 M NaCl and 20 mM imidazole were added) for nickel chelate column binding was added. The mixture was passed through a 100-ml nickel chelate column equilibrated with a buffer for nickel chelate column binding. Then, the column was washed with a suitable amount of a buffer for nickel chelate column binding to remove a degraded collagenase that were not able to be adsorbed to the nickel chelate column. Thereafter, 100 ml of a buffer for nickel chelate column binding to which 500 mM imidazole had been added was passed through the column. Thus, the fusion collagenase G having the CBD was collected.

(2-4) Confirmation of Removal of Degraded Collagenase

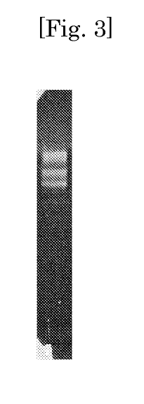

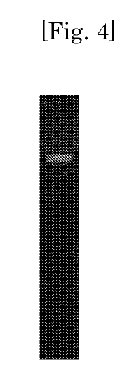

To confirm that the degraded collagenase was removed from the extract of the fusion collagenase G, the activity staining was performed on the extract of the E. coli expressing the fusion collagenase G and the solution of the fusion collagenase G subjected to the affinity chromatography. The activity staining was performed on 0.25 μl of the extract of the E. coli expressing the fusion collagenase G obtained in (2-2) and 2.5 μl of the solution of the fusion collagenase G subjected to the affinity chromatography obtained in (2-3) with Zymogram-PAGE mini (manufactured by TEFCO). As a result, five bands indicating protease activity were observed for the extract of the E. coli expressing the fusion collagenase G (FIG. 3). On the other hand, for the solution of the fusion collagenase G subjected to the affinity chromatography, one band indicating the maximum molecular weight was observed as a main band among the above five bands (FIG. 4). Based on the comparison and the analysis of these bands, it was found that part or all of the CBD was degraded when the fusion collagenase G was expressed by E. coli. The foregoing results showed that the fusion collagenase having the CBD was successfully selectively collected by purifying the extract of the E. coli expressing the fusion collagenase G by the affinity chromatography and thereby removing the collagenase in which part or all of the CBD was degraded.

### Example 3

### Expression of Fusion Collagenase in which Histidine Tag is Linked to Carboxyl Terminal of Collagenase H Derived from Clostridium histolyticum

(3-1) Preparation of Collagenase H Gene Fragment

A collagenase H gene derived from Clostridium histolyticum was amplified from the 5′ end to the 3′ end thereof by PCR with a template of a genomic DNA derived from Clostridium histolyticum. In this case, the primers were designed so that the 3′ end of the amplified collagenase H gene served as an XbaI-recognition sequence and further that a BamHI-recognition sequence was added subsequent to the stop codon of the gene. As a result, a mutation was introduced into the amino acid sequence at one position (an amino acid at position 980 of SEQ ID NO: 5) on the carboxyl terminal side of the amino acid sequence of natural collagenase H described in Non-Patent Literature 2. Thus, the amino acid sequence was modified to the amino acid sequence of SEQ ID NO: 6.

The primers used in this PCR were as follows.

| colH-F: | |
|---|---|
| ATGAAAAGGAAATGTTTATC | (SEQ ID NO: 15) |
| colH-R: | |
| CCGGATCCTATCTAGATACTGAACCTT | (SEQ ID NO: 16) |

The amplified collagenase H gene fragment was digested with BamHI.

(3-2) Preparation of DNA Fragment Including Region Encoding Histidine Tag

A DNA fragment including the region encoding the histidine tag was prepared by the same method as in Example 1.

(3-3) Preparation of lacZ Promoter Fragment

A lacZ promoter fragment was prepared by the same method as in Example 1.

(3-4) Construction of Expression Vector for Fusion Collagenase H to which Histidine Tag is Linked

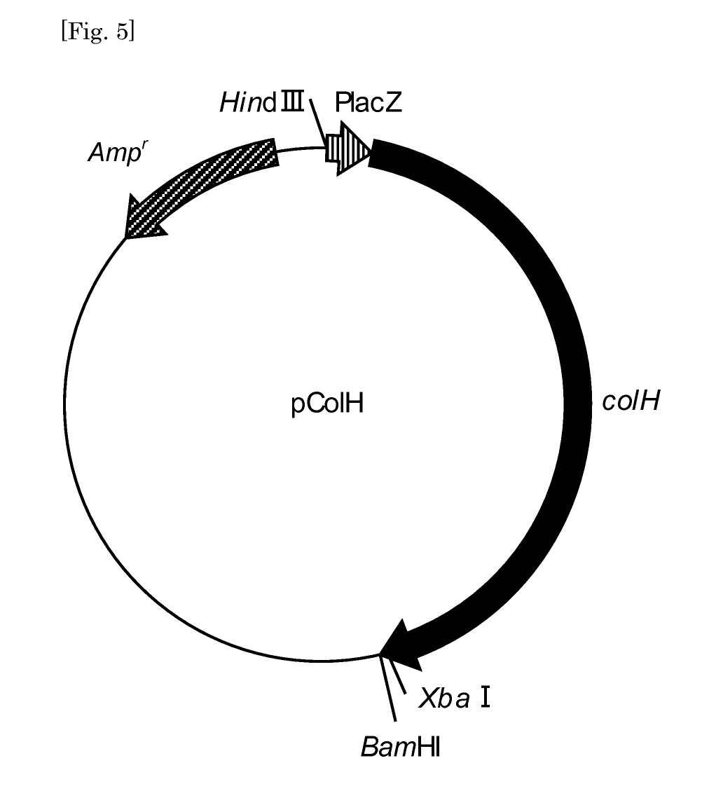

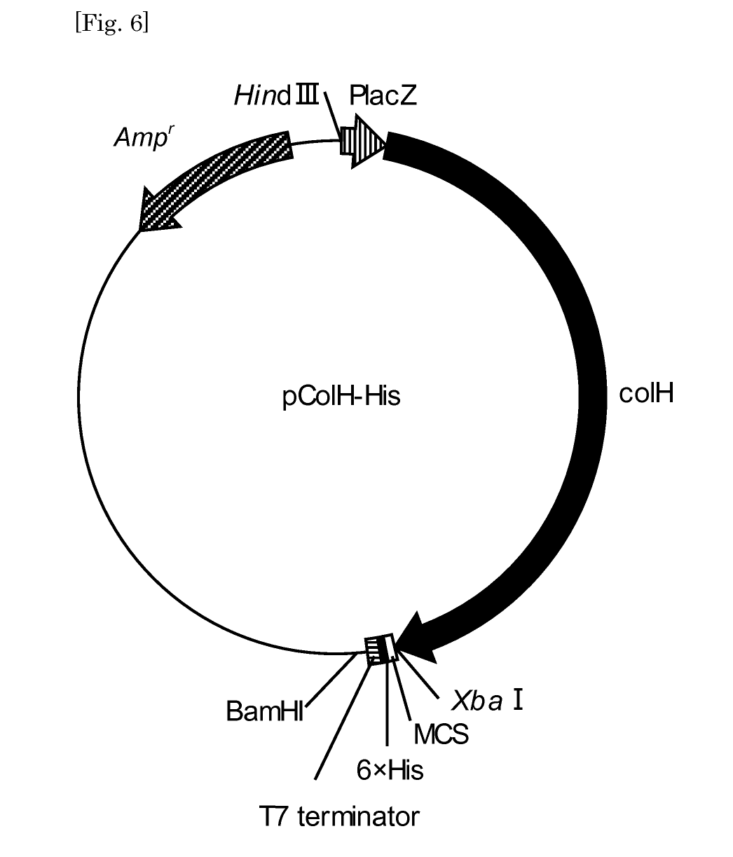

Three kinds of DNA fragments prepared by the method described above were inserted into pBR322, a commercially available vector, so as to link them in order of the lacZ promoter, the collagenase H gene, and the DNA fragment including the histidine tag from the 5′ end. First, to insert the lacZ promoter and the collagenase H gene into pBR322, the lacZ promoter region and the collagenase H gene fragment prepared as described above were phosphorylated, and then inserted into pBR322 that had been double-digested with HindIII and BamHI to construct pColH (FIG. 5). It is to be noted that the lacZ gene promoter region and the collagenase H gene were linked to each other at their blunt ends. Subsequently, the DNA fragment including the DNA encoding the histidine tag prepared by the method described above was inserted into pColH that had been double-digested with XbaI and BamHI to construct pColH-His (FIG. 6). The DNA that was finally inserted into pBR322 was a DNA having the base sequence from positions 1 to 3105 of Sequence Listing: 8.

(3-5) Preparation of E. coli Expressing Fusion Collagenase H

E. coli expressing the fusion collagenase H was created by the same method as in Example 1.

### Example 4

### Culturing of E. coli Expressing Fusion Collagenase H, and Selective Collection of Collagenase H Having CBD

(4-1) Culturing of E. coli Expressing Fusion Collagenase H

The E. coli expressing the fusion collagenase H obtained in Example 3 was cultured by the method described in Example 2 to obtain a culture solution.

(4-2) Preparation of Extract of E. coli Expressing Fusion Collagenase H

An extract of the E. coli expressing the fusion collagenase H was obtained from the resulting culture solution obtained in (4-1) by the method described in Example 2.

(4-3) Selective Collection of Fusion Collagenase H Having CBD by Affinity Chromatography

The extract of the E. coli expressing the fusion collagenase H obtained in (4-2) was subjected to affinity chromatography by the method described in Example 2 to thus collect the fusion collagenase H having the CBD.

(4-4) Confirmation of Removal of Degraded Collagenase

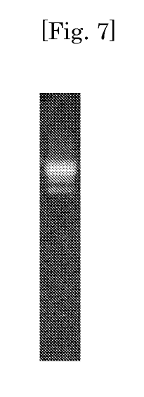

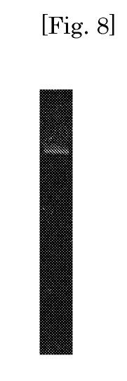

To confirm how much the degraded collagenase was removed from the fusion collagenase H, the activity staining was performed after electrophoresis by the method described in Example 2. As a result, four bands indicating protease activity were observed for the extract of the E. coli expressing the fusion collagenase H (FIG. 7). On the other hand, for the solution of the fusion collagenase H subjected to the affinity chromatography, one band indicating the maximum molecular weight was observed as a main band among the above four bands (FIG. 8). Based on the comparison and the analysis of these bands, it was found that part or all of the CBD in the fusion collagenase H expressed by E. coli was degraded. The foregoing results showed that the fusion collagenase H having the CBD was successfully selectively collected by purifying the extract of the E. coli expressing the fusion collagenase H by the affinity chromatography and thereby removing the collagenase in which part or all of the CBD was degraded.

### INDUSTRIAL APPLICABILITY

According to the present invention, a collagenase as a recombinant protein can be produced with high purity and without including a degraded product caused by an action of a host. The use of a collagenase produced by the method of the present invention allows, for example, pancreatic islets to be separated from a pancreas without decreasing the quality of the pancreatic islets, thereby greatly contributing to pancreatic islet transplantation to diabetic patients.

1.-18. (canceled)

19. A method for producing a fusion collagenase, comprising:
a culture obtained by culturing E. coli transformed by any one of a DNA and an expression vector comprising the DNA is purified by affinity chromatography corresponding to an affinity tag to thereby selectively collect the fusion collagenase having a collagen-binding domain, wherein the DNA encodes the fusion collagenase in which the affinity tag is directly or indirectly linked to a collagenase in such a manner that a fragment having a collagenase activity and the affinity tag are separated from each other when the fusion collagenase expressed in E. coli is degraded by an action of E. coli.

20. The method for producing according to claim 19, wherein the fusion collagenase is a fusion collagenase in which the affinity tag is directly or indirectly linked to a carboxyl terminal of the collagenase.

21. The method for producing according to claim 20, wherein the fusion collagenase is a fusion collagenase in which the affinity tag is two or more consecutive histidine residues.

22. The method for producing according to claim 19, wherein the fusion collagenase is derived from Clostridium histolyticum.

23. The method for producing according to claim 19, wherein the collagenase is selected from the following (i), (ii), (iii) and (iv):
(i) a collagenase comprising all or part of an amino acid sequence from positions −110 to 1008 of any one of SEQ ID NOS: 1 and 2;
(ii) a collagenase comprising an amino acid sequence from positions −110 to 1008 of any one of SEQ ID NOS: 1 and 2 in which one or more amino acids are deleted, substituted, inserted, or added;
(iii) a collagenase comprising an amino acid sequence having a homology of 70% or more with the amino acid sequence from positions −110 to 1008 of any one of SEQ ID NOS: 1 and 2; and
(iv) a collagenase in which all or part of an amino acid sequence from positions −110 to −1 of any one of SEQ ID NOS: 1 and 2 is removed from any one of the collagenases described in (i) to (iii).

24. The method for producing according to claim 23, wherein the fusion collagenase comprises all or part of an amino acid sequence from positions −110 to 1021 of SEQ ID NO: 3.

25. The method for producing according to claim 24, wherein the fusion collagenase is a fusion collagenase from which all or part of an amino acid sequence from positions −110 to −1 of SEQ ID NO: 3 is removed.

26. The method for producing according to claim 19, wherein the collagenase is selected from the following (i), (ii), (iii) and (iv):
(i) a collagenase comprising all or part of an amino acid sequence from positions −40 to 981 of any one of SEQ ID NOS: 5 and 6;
(ii) a collagenase comprising an amino acid sequence from positions −40 to 981 of any one of SEQ ID NOS: 5 and 6 in which one or more amino acids are deleted, substituted, inserted, or added;
(iii) a collagenase comprising an amino acid sequence having a homology of 70% or more with the amino acid sequence from positions −40 to 981 of any one of SEQ ID NOS: 5 and 6; and
(iv) a collagenase in which all or part of an amino acid sequence from positions −40 to −1 of any one of SEQ ID NOS: 5 and 6 is removed from any one of the collagenases described in (i) to (iii).

27. The method for producing according to claim 26, wherein the fusion collagenase comprises all or part of an amino acid sequence from positions −40 to 994 of SEQ ID NO: 7.

28. The method for producing according to claim 27, wherein the fusion collagenase is a fusion collagenase from which all or part of an amino acid sequence from positions −40 to −1 of SEQ ID NO: 7 is removed.

29. The method for producing according to claim 19, wherein the DNA encoding the fusion collagenase is a DNA comprising a base sequence from positions 1 to 3396 of SEQ ID NO: 4.

30. The method for producing according to claim 19, wherein the DNA encoding the fusion collagenase is a DNA comprising a base sequence from positions 1 to 3105 of SEQ ID NO: 8.


SEQUENCE LISTING (16 sequences)

SEQ ID NO: 1 - protein, 1118 aa, Clostridium histolyticum
  SIGNAL (-110)..(-1)
  mat_peptide (1)..(1008)
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 2 - protein, 1118 aa, Clostridium histolyticum
  SIGNAL (-110)..(-1)
  mat_peptide (1)..(1008)
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVSR

SEQ ID NO: 3 - protein, 1131 aa, Clostridium histolyticum
  SIGNAL (-110)..(-1)
  mat_peptide (1)..(1021)
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVSRKLAAALEHHHHHH

SEQ ID NO: 4 - DNA, 3396 bp, Clostridium histolyticum
  CDS (1)..(3396)
atgaaaaaaaatattttaaagattcttatggatagttattctaaagaatctaaaattcaaactgtacgtagggttacgagtgtatcacttttagcggtatatcttactatgaatacttcaagtttagttttagcaaaaccaatagaaaatactaatgatactagtataaaaaatgtggagaaattaagaaatgctccaaatgaagagaatagtaaaaaggtagaagatagtaaaaatgataaggtagaacatgtgaaaaatatagaagaggcaaaggttgagcaagttgcacccgaagtaaaatctaaatcaactttaagaagtgcttctatagcgaatactaattctgagaaatatgattttgagtatttaaatggtttgagctatactgaacttacaaatttaattaaaaatataaagtggaatcaaattaatggtttatttaattatagtacaggttctcaaaagttctttggagataaaaatcgtgtacaagctataattaatgctttacaagaaagtggaagaacttacactgcaaatgatatgaagggtatagaaactttcactgaggttttaagagctggtttttatttagggtactataatgatggtttatcttatttaaatgatagaaacttccaagataaatgtatacctgcaatgattgcaattcaaaaaaatcctaactttaagctaggaactgcagttcaagatgaagttataacttctttaggaaaactaataggaaatgcttctgctaatgctgaagtagttaataattgtgtaccagttctaaaacaatttagagaaaacttaaatcaatatgctcctgattacgttaaaggaacagctgtaaatgaattaattaaaggtattgaattcgatttttctggtgctgcatatgaaaaagatgttaagacaatgccttggtatggaaaaattgatccatttataaatgaacttaaggccttaggtctatatggaaatataacaagtgcaactgagtgggcatctgatgttggaatatactatttaagtaaattcggtctttactcaactaaccgaaatgacatagtacagtcacttgaaaaggctgtagatatgtataagtatggtaaaatagcctttgtagcaatggagagaataacttgggattatgatgggattggttctaatggtaaaaaggtggatcacgataagttcttagatgatgctgaaaaacattatctgccaaagacatatacttttgataatggaacctttattataagagcaggggataaggtatccgaagaaaaaataaaaaggctatattgggcatcaagagaagtgaagtctcaattccatagagtagttggcaatgataaagctttagaggtgggaaatgccgatgatgttttaactatgaaaatatttaatagcccagaagaatataaatttaataccaatataaatggtgtaagcactgataatggtggtctatatatagaaccaagagggactttctacacttatgagagaacacctcaacaaagtatatttagtcttgaagaattgtttagacatgaatatactcactatttacaagcgagatatcttgtagatggtttatgggggcaaggtccattttatgaaaaaaatagattaacttggtttgatgaaggtacagctgaattctttgcaggatctacccgtacatctggtgttttaccaagaaaatcaatattaggatatttggctaaggataaagtagatcatagatactcattaaagaagactcttaattcagggtatgatgacagtgattggatgttctataattatggatttgcagttgcacattacctatatgaaaaagatatgcctacatttattaagatgaataaagctatattgaatacagatgtgaaatcttatgatgaaataataaaaaaattaagtgatgatgcaaataaaaatacagaatatcaaaaccatattcaagagttagcagataaatatcaaggagcaggcatacctctagtatcagatgattacttaaaagatcatggatataagaaagcatctgaagtatattctgaaatttcaaaagctgcttctcttacaaacactagtgtaacagcagaaaaatctcaatattttaacacattcactttaagaggaacttatacaggtgaaacttctaaaggtgaatttaaagattgggatgaaatgagtaaaaaattagatggaactttggagtcccttgctaaaaattcttggagtggatacaaaactttaacagcatactttacgaattatagagttacaagcgataataaagttcaatatgatgtagttttccatggggttttaacagataatgcggatattagtaacaataaggctccaatagcaaaggtaactggaccaagcactggtgctgtaggaagaaatattgaatttagtggaaaagatagtaaagatgaagatggtaaaatagtatcatatgattgggattttggcgatggtgcaactagtagaggcaaaaattcagtacatgcttacaaaaaagcaggaacatataatgttacattaaaagtaactgacgataagggtgcaacagctacagaaagctttactatagaaataaagaacgaagatacaacaacacctataactaaagaaatggaacctaatgatgatataaaagaggctaatggtccaatagttgaaggtgttactgtaaaaggtgatttaaatggttctgatgatgctgataccttctattttgatgtaaaagaagatggtgatgttacaattgaacttccttattcagggtcatctaatttcacatggttagtttataaagagggagacgatcaaaaccatattgcaagtggtatagataagaataactcaaaagttggaacatttaaatctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtatctagaaagcttgcggccgcactcgagcaccaccaccaccaccactga

SEQ ID NO: 5 - protein, 1021 aa, Clostridium histolyticum
  SIGNAL (-40)..(-1)
  mat_peptide (1)..(981)
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 6 - protein, 1021 aa, Clostridium histolyticum
  SIGNAL (-40)..(-1)
  mat_peptide (1)..(981)
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVSR

SEQ ID NO: 7 - protein, 1034 aa, Clostridium histolyticum
  SIGNAL (-40)..(-1)
  mat_peptide (1)..(994)
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVSRKLAAALEHHHHHH

SEQ ID NO: 8 - DNA, 3105 bp, Clostridium histolyticum
  CDS (1)..(3105)
atgaaaaggaaatgtttatctaaaaggcttatgttagctataacaatggctacaatatttacagtgaacagtacattaccaatttatgcagctgtagataaaaataatgcaacagcagctgtacaaaatgaaagtaagaggtatacagtatcatatttaaagactttaaattattatgacttagtagatttgcttgttaagactgaaattgagaatttaccagacctttttcagtatagttcagatgcaaaagagttctatggaaataaaactcgtatgagctttatcatggatgaaattggtagaagggcacctcagtatacagagatagatcataaaggtattcctactttagtagaagttgtaagagctggattttacttaggattccataacaaggaattgaatgaaataaacaagaggtcttttaaagaaagggtaataccttctatattagcaattcaaaaaaatcctaattttaaactaggtactgaagttcaagataaaatagtatctgcaacaggacttttagctggtaatgaaacagcgcctccagaagttgtaaataattttacaccaatacttcaagactgtataaagaatatagacagatacgctcttgatgatttaaagtcaaaagcattatttaatgttttagctgcacctacctatgatataactgagtatttaagagctactaaagaaaaaccagaaaacactccttggtatggtaaaatagatgggtttataaatgaacttaaaaagttagctctttatggaaaaataaatgataataactcttggataatagataacggtatatatcatatagcacctttagggaagttacatagcaataataaaataggaatagaaactttaacagaggttatgaaagtttatccttatttaagtatgcaacatttacaatcagcagatcaaattaagcgtcattatgattcaaaagatgctgaaggaaacaaaatacctttagataagtttaaaaaggaaggaaaagaaaaatactgtccaaaaacttatacatttgatgatggaaaagtaataataaaagctggtgctagagtagaagaagaaaaagttaaaagactatactgggcatcaaaggaagttaactctcaattctttagagtatacggaatagacaaaccattagaagaaggtaatccagatgatatattaacaatggttatctacaacagtcccgaagaatataaactcaatagtgttctatacggatatgatactaataatggtggtatgtatatagagccagaaggaactttcttcacctatgaaagagaagctcaagaaagcacatacacattagaagaattatttagacatgaatatacacattatttgcaaggaagatatgcagttccaggacaatggggaagaacaaaactttatgacaatgatagattaacttggtatgaagaaggtggagcagaattatttgcaggttctactagaacttctggaatattaccaagaaagagtatagtatcaaatattcataatacaacaagaaataatagatataagctttcagacactgtacattctaaatatggtgctagttttgaattctataattatgcatgtatgtttatggattatatgtataataaagatatgggtatattaaataaactaaatgatcttgcaaaaaataatgatgttgatggatatgataattatattagagatttaagttctaattatgctttaaatgataaatatcaagatcatatgcaggagcgcatagataattatgaaaatttaacagtgccttttgtagctgatgattatttagtaaggcatgcttataagaaccctaatgaaatttattctgaaatatctgaagtagcaaaattaaaggatgctaagagtgaagttaagaaatcacaatattttagtacctttactttgagaggtagttacacaggtggagcatctaaggggaaattagaagatcaaaaagcaatgaataagtttatagatgattcacttaagaaattagatacgtattcttggagtgggtataaaactttaactgcttatttcactaattataaagttgactcttcaaatagagttacttatgatgtagtattccacggatatttaccaaacgaaggtgattccaaaaattcattaccttatggcaagatcaatggaacttacaagggaacagagaaagaaaaaatcaaattctctagtgaaggctctttcgatccagatggtaaaatagtttcttatgaatgggatttcggagatggtaataagagtaatgaggaaaatccagagcattcatatgacaaggtaggaacttatacagtgaaattaaaagttactgatgacaagggagaatcttcagtatctactactactgcagaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtatctagaaagcttgcggccgcactcgagcaccaccaccaccaccactga

SEQ ID NO: 9 - DNA, 25 bp, Artificial Sequence
  Synthetic oligonucleotide primer
atgaaaaaaaatattttaaagattc

SEQ ID NO: 10 - DNA, 27 bp, Artificial Sequence
  Synthetic oligonucleotide primer
ccggatcctatctagatacccttaact

SEQ ID NO: 11 - DNA, 28 bp, Artificial Sequence
  Synthetic oligonucleotide primer
gctctagaaagcttgcggccgcactcga

SEQ ID NO: 12 - DNA, 24 bp, Artificial Sequence
  Synthetic oligonucleotide primer
cgggatccggatatagttcctcct

SEQ ID NO: 13 - DNA, 28 bp, Artificial Sequence
  Synthetic oligonucleotide primer
ccggcaagcttgcccaatacgcaaaccg

SEQ ID NO: 14 - DNA, 18 bp, Artificial Sequence
  Synthetic oligonucleotide primer
agctgtttcctgtgtgaa

SEQ ID NO: 15 - DNA, 20 bp, Artificial Sequence
  Synthetic oligonucleotide primer
atgaaaaggaaatgtttatc

SEQ ID NO: 16 - DNA, 27 bp, Artificial Sequence
  Synthetic oligonucleotide primer
ccggatcctatctagatactgaacctt